(19) United States
(12) Patent Application Publication
(10) Pub. No.: US 2022/0110627 A1
(43) Pub. Date: Apr. 14, 2022

(54) SURGICAL KNOT ASSEMBLY FOR TYING A SURGICAL KNOT

(71) Applicant: MatrixLabs Medical Co., Ltd., New Taipei City (TW)

(72) Inventors: Fungchao Tu, New Taipei City (TW); WenYih Wu, Taipei City (TW); Kuei-Hua Chen, Kaohsiung City (TW)

(21) Appl. No.: 17/070,181

(22) Filed: Oct. 14, 2020

(51) Int. Cl. A61B 17/04

(52) U.S. Cl. CPC A61B 17/0469; A61B 2017/0475; A61B 2017/0474

(57) ABSTRACT

A surgical knot assembly for tying a surgical knot is disclosed. The surgical knot assembly for tying a surgical knot includes an outer tube, an inner tube slidably interconnected within the outer tube and having a distal end protruding from the outer tube as a neck and a strand of suture material having a portion of a length configured as pre-tied knot and a loop. The loop is adapted to receive at least a strand of suture filaments therein, and fastening of the loop, the pre-tied knot and the suture filament to form a surgical knot. While the inner tube is retracted, the loop is closed to hold the at least one suture material and receive the at least one suture filament to form the surgical knot successfully.

### FIELD OF INVENTION

The present disclosure relates to a surgical knot assembly, and more particularly to the disclosure related to a surgical knot assembly adapted for facilitating the process of suturing and knot-tying in a surgical operation.

### BACKGROUND OF THE INVENTION

To secure a suture extending through an object, a knot is normally formed on a distal end of the suture. For example, in laparoscopic surgery, the tying of sutures and placement of a surgical knot for closure and treatment of a wound or an opening can be challenging for many surgeons. A surgeon performing suturing and knot-tying in intracorporeal surgery may encounter with more obstacles than performing the operation in an open surgical procedure because surgical instruments are restricted in a confined space and operative field, and limited visibility as the surgeon is unable to view the site of the surgery directly, but instead must rely upon images on a monitor which significantly affects hand-eye coordination and manual dexterity.

Further, conventional intracorporeal suturing and knot-tying process requires the surgeon to manoeuvre a needle and suture material in a particular number of steps, directions and positionings of the suture material when forming the knot. And generally, is required to repeat the process over several times to deliver multiple throws for knot security which adequately tightens and supports the wound or opening site with sufficient tension strength. Some examples of the conventional intracorporeal suturing and knot-tying techniques include a square knot or surgeon's knot, but these techniques can be exceedingly difficult, complex and time-consuming. Therefore, the way for rapidly and efficiently forming a knot for surgical suture is eagerly needed. It is to be understood that, if any prior art information is referred to herein, such reference does not constitute an admission that the information forms part of the common general knowledge in the art.

### SUMMARY OF THE INVENTION

To solve the aforementioned problems of the conventional way for forming a knot, especially for the surgical knot performed in a surgical operation, the main objective of the invention is to provide a knotting assembly that forms a pre-knot structure for rapidly and conveniently tie a knot with sutures.

According to a first aspect of the present disclosure, a surgical knot assembly for tying a surgical knot, comprising: an outer tube; an inner tube slidably interconnected within the outer tube and having a distal end protruding from the outer tube as a neck; and a strand of suture material having a first portion of a length configured releasably engaged to the neck being a pre-tied knot, and having a second portion of a length extending from a cavity at the distal end of the inner tube, thereby forming a loop between the distal end of the inner tube and the neck to receive at least a strand of suture filament therein, wherein size of the loop is variable by manoeuvring the suture material and closing the loop as the neck retracts back into the outer tube, allowing the loop, the pre-tied knot and the suture filament to be fastened together forming a surgical knot held at the cavity.

Preferably, the pre-tied knot is whirled around the inner tube at least twice and the suture filament intertwined with the loop at least twice when forming the surgical knot.

More preferably, the suture material and the suture filament are compressed between 3% to 30% when forming the surgical knot.

The present invention could be easily performed by cooperating the pre-tied knot, the loop and the suture filament f to ensure the surgical knot assembly is efficient for facilitating the formation of a secure surgical knot. The present disclosure seeks to provide a surgical knot assembly that will overcome or substantially ameliorate at least one or more of the deficiencies of a prior art, or to at least provide an alternative.

### BRIEF DESCRIPTION OF THE DRAWINGS

FIG. 1 is a perspective view of a first embodiment of a surgical knot assembly in accordance with the present disclosure.

FIGS. 2 to 4 depicts the surgical knot assembly of FIG. 1 in operation.

FIG. 5 is a perspective view of a second embodiment of a surgical knot assembly in accordance with the present disclosure.

FIGS. 6 to 8 depicts the surgical knot assembly of FIG. 5 in operation.

FIG. 9 is a perspective view of a third embodiment of a surgical knot assembly in accordance with the present disclosure.

FIG. 10 is a perspective view of a fourth embodiment of a surgical knot assembly in accordance with the present disclosure.

FIGS. 11 to 15 depict a cross-sectional view of the surgical knot inside a cavity of an inner tube in different embodiments in accordance with the present disclosure.

FIG. 16 is a cross-sectional view of the compressed surgical knot showing sides with curved edges abutting the inner cavity of an inner tube in accordance with the present disclosure in one of the preferred embodiments.

FIG. 17 is a cross-sectional view of the compressed surgical knot in accordance with the present disclosure in another preferred embodiments.

### DETAILED DESCRIPTION OF THE PREFERRED EMBODIMENTS

Other objects, advantages and novel features of the invention will become more apparent from the following detailed description when taken in conjunction with the accompanying drawings.

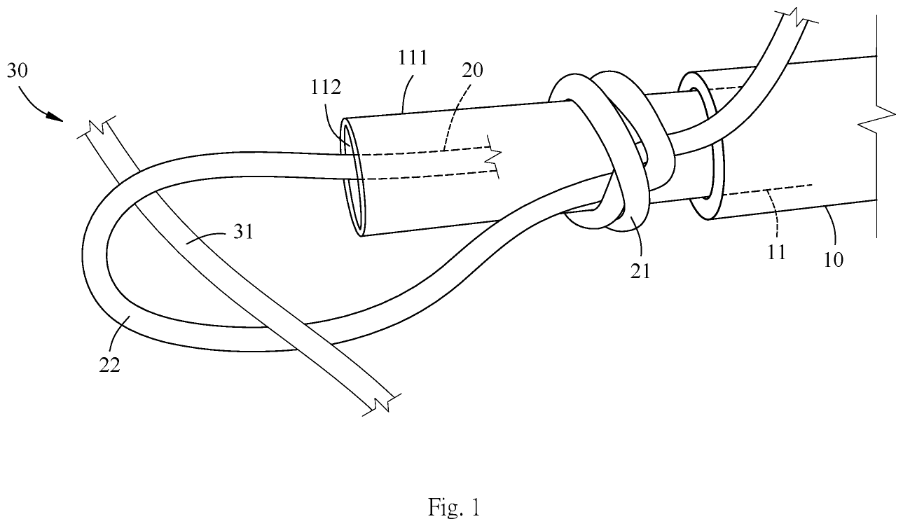

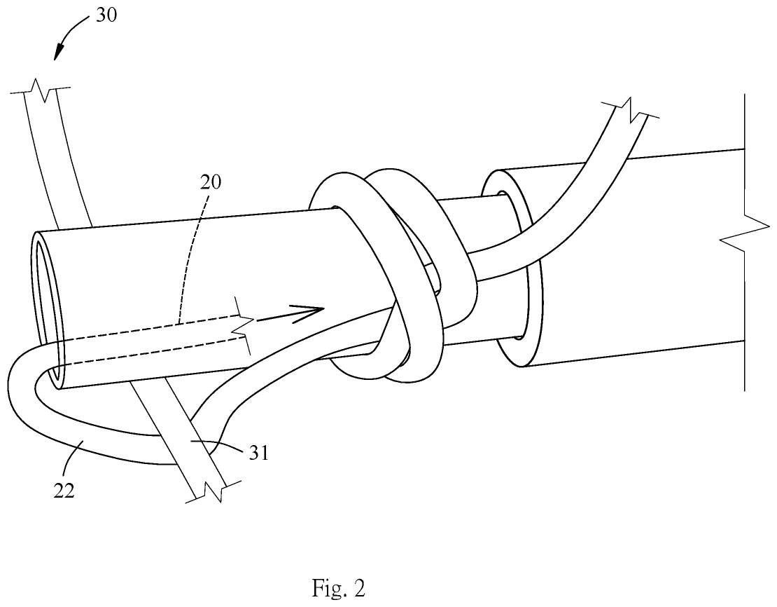

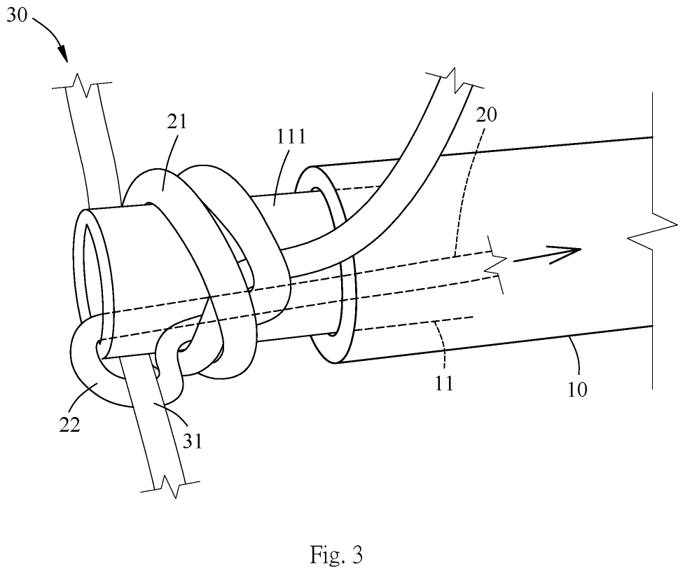

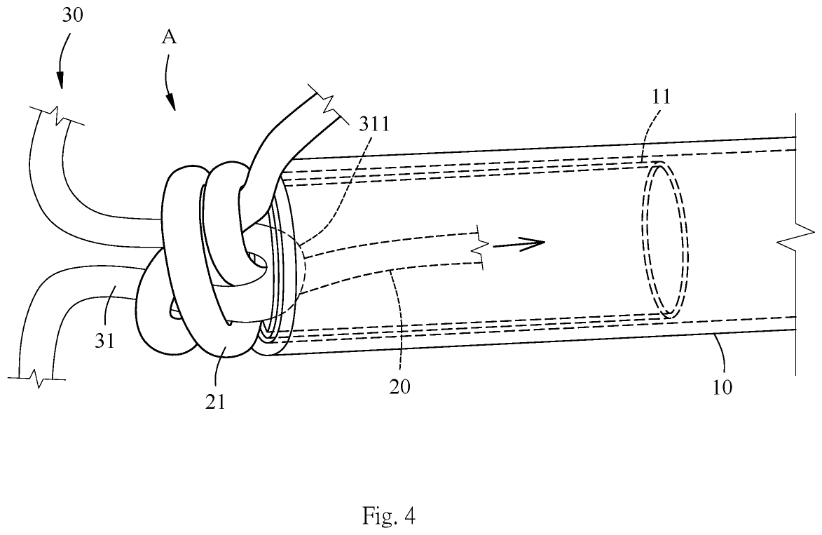

Referring to FIGS. 1-4, a surgical knot assembly is disclosed. The surgical knot assembly for tying a surgical knot comprises an outer tube 10, an inner tube 11 slidably interconnected within the outer tube 10 and having a distal end protruding from the outer tube 10 as a neck 111, and a strand of suture material 20 having a first portion of a length configured releasably engaged to the neck 111 being a pre-tied knot 21, and having a second portion of a length extending from a cavity 112 at the distal end of the inner tube 11, thereby forming a loop 22 between the distal end of the inner tube 11 and the neck 111 to receive at least a strand of suture filament 30 therein, and size of the loop 22 is variable by manoeuvring the suture material 20 and closing the loop 22 as the neck 111 retracts back into the outer tube 10 as shown in, allowing the loop 22, the pre-tied knot 21 and the suture filament 30 to be fastened together forming a surgical knot A held at the cavity 112. The surgical knot assembly is readily detachable from each other, allowing an operator to replace either of such parts if required (not shown). In some embodiments, the formed knot may have a variable knot strength between 1.8 and 3.5 kilograms (kg). The outer tube 10 is fabricated from a length of tube having a diameter between 1.00 to 4.00 mm, and a length between 5 and 40 mm. The inner tube 11 is fabricated from a length of tube having a diameter between 0.50 and 2.50 mm.

In the first embodiment of the present invention, the suture filament 30 and the suture material 20 are surgical suture shared a same size, specification or even a same brand. However, the present invention does not intend to be limited in this particular use of surgical suture. Other applications applied with same or similar knotting technique should be considered disclosed and protected by the present invention. The relative position between the outer tube 10, inner tube 11 and pre-tied knot 21 is variable subject to the working and fastening a portion of the suture filament 31 and can be fixed during the operation by the operator to ensure that the suturing and knot-tying process for a patient can be adequately tended. After the portion of the suture filament 31 is received by the loop 22, as the operator retracts the inner tube 11, the surgical knot A is partially formed and can be identified at the tip of the cavity 112. Meanwhile, the portion of the suture filament 31 will be bended or folded and tighten by the pre-tied knot 21 to form the surgical knot A and identified at the tip of the cavity 112. After that, it is optional to trim the remaining filament of the suture filament 30 or the suture material 20 excesses from the surgical knot A.

The cavity 112 provides holding and limitation of movements for the partially formed surgical knot A to prevent disengagement of the suture filament 30 before the surgical knot A is fully tighten. Further, in the process of tightening, it is important to make sure that the portion of the suture filament 31 is slide inside the cavity 112 with at least its tip 311. The resulting gives a feedback mechanism allowing the operator to be alerted and maintain a tactile feel regarding the location and status of the partially formed surgical knot A as being tied. After the surgical knot A is fully and successfully tighten, it should be secure at the opening of the cavity 112 without slide inside the cavity 112. To prevent failing the knotting process if the surgical knot A slide into the cavity 112 occur, the such surgical knot A should be tighten with its size larger than the opening of the cavity 112.

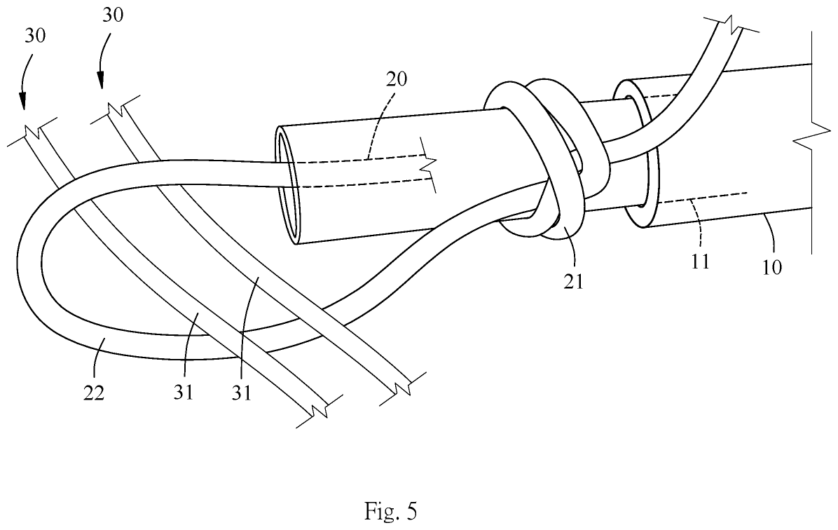

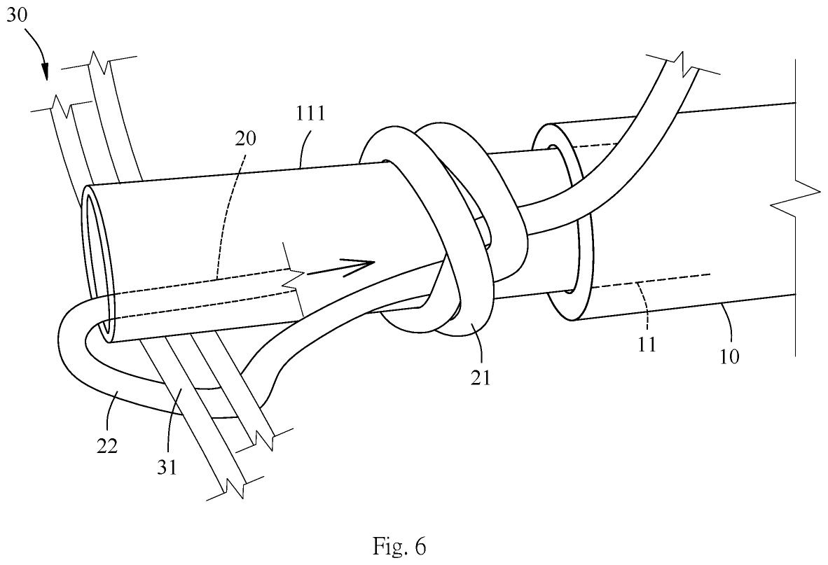

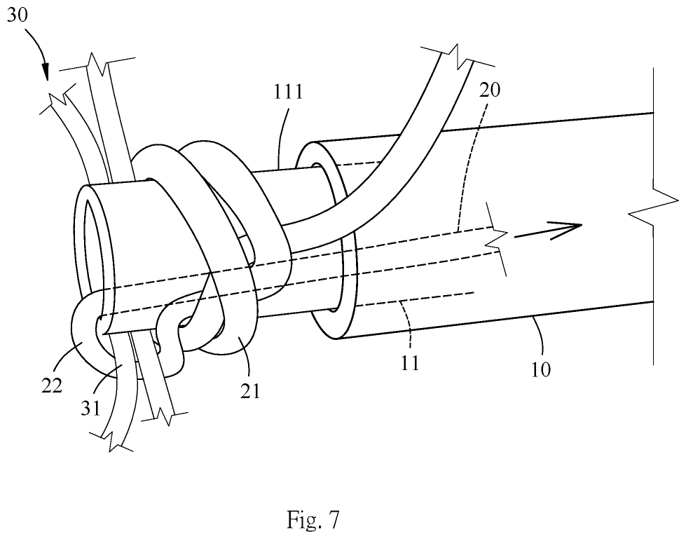

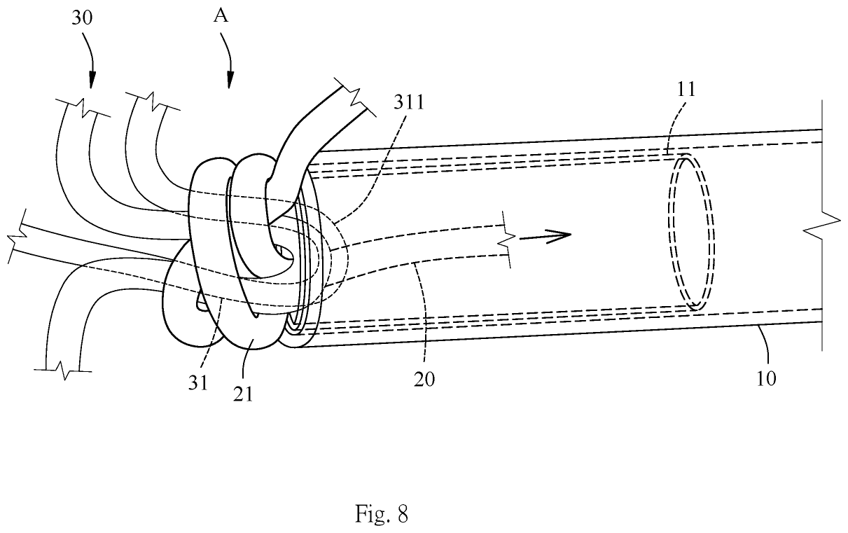

Referring to FIGS. 5-8, a second preferred embodiment of the present invention is disclosed. The suture material 20 is manoeuvred to close the loop 22. Despite one strand of suture filaments for forming the surgical knot A, the loop 22 could receive two or more strands of suture filament 30 therein for fastening of the surgical knot A in this embodiment.

In this preferred embodiment of the present invention, as two strands of suture filaments 30 received by the loop 22, the inner tube 11 could be retracted from the outer tube 10 making the pre-tied knot 20 departed from the neck 111, the surgical knot A will then partially formed and can be identified at the tip of the cavity 112. Meanwhile, the portion of two suture filaments 31 will be bended or folded and tighten by the pre-tied knot 21 to form the surgical knot A and identified at the tip of the cavity 112 just like the first embodiment descript above.

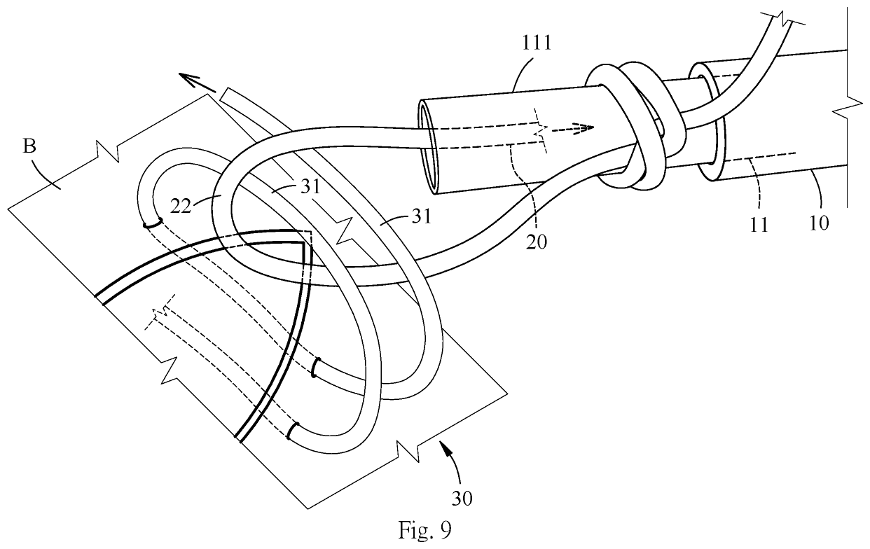

In certain embodiment, the suture filaments 30 can be passed the loop 22 several times in view of the treatment required at a wound or surgical site B, as shown in FIG. 9. By this practice, two strands of suture filaments 30 are originated from the same suture filament 30 repeated looping from the wound or surgical site B.

In this preferred embodiment, as two strands of suture filaments 30 from the wound or surgical site B received by the loop 22, the inner tube 11 could be retracted from the outer tube 10 making the pre-tied knot 20 departed from the neck 111, the surgical knot A will then partially formed and can be identified at the tip of the cavity 112. Meanwhile, the portion of two suture filaments 31 will be bended or folded and tighten by the pre-tied knot 21 to form the surgical knot A and identified at the tip of the cavity 112 just like the first embodiment descript above.

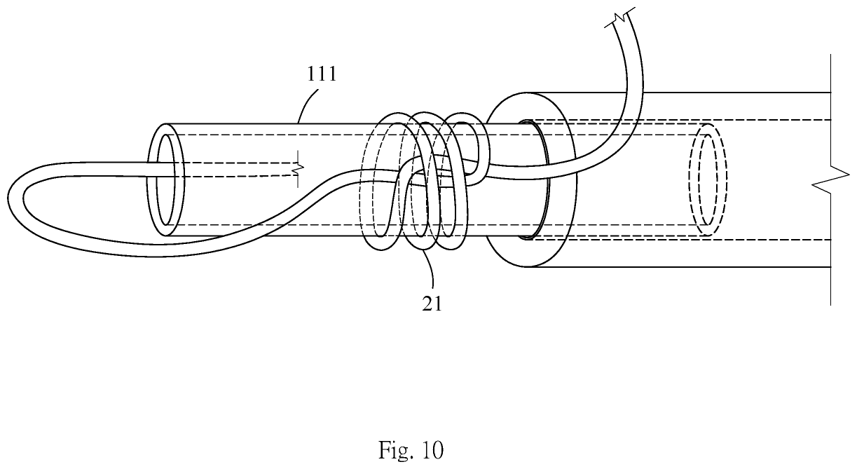

Yet in certain embodiment, the pre-tied knot 21 can be whirled around the inner tube 11 once or twice and having the suture filaments 30 passing the loop 22 several times in view of the treatment required at the wound or surgical site B, as shown in FIG. 10. In some embodiments, the pre-tied knot 21 is whirled multiple times around the inner tube 11. The number of times that the pre-tied knot 21 is whirled around the inner tube 11 is subject to the suture material 20 used, number of strands of the suture filament 30 applied, and the desired knot strength that the operator sees fit for the operation. In one embodiment, if the diameters of the suture material 20 and the suture filament 30 between 0.350 and 0.399 mm was adopted, wherein the suture filament 30 intertwined with the loop 22 at least twice when forming the surgical knot, as shown in FIG. 9. In accordance, the knot strength obtained is approximately 2.4 kilograms (kg).

Furthermore, to avoid the pre-tied knot 21 loosen during retraction of the inner tube 11, an end of the suture material 20 can be attached or even temperately adhered to the outer tube 10. More preferably, a tension could be retained between the suture material 20 and the outer tube 10. For example, the suture material 20 and the outer tube 10 could be retained or secured by a resilient element or an elastic element to provide a tension therebetween. In some preferred embodiment, the resilient element or the elastic element may be a spring, a silicon thread or a rubber thread put on the outer tube 10 creating the said tension for the suture material 20. As the suture material 20 being retained by the tension, the knotting process could be performed more easily and also reduced the possibility of loosening the surgical knot A after tightening.

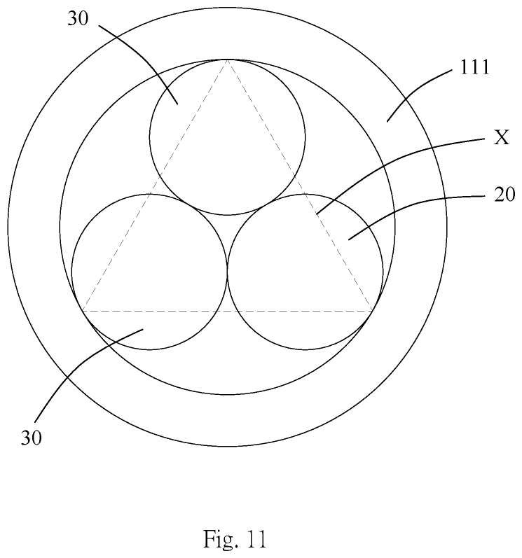

Multiple types of cross-sectional views of the suture material 20 and the portion of the suture filament 31 at the cavity 112 after the surgical knot A formed are provided in different embodiments. It is understandable or reasonable, to hold the suture material 20 and the portion of the suture filament 31 within the inner tube 11, an inner diameter of the inner tube 11 is preferred to be larger than all outer diameters of the suture material 20 and the portion of the suture filament 31 combined. As shown in FIG. 11, is that the cross-sectional view of the suture material 20 and the portion of the suture filament 31 is geometrically shaped X. The cross-sectional view is polygonally shaped at the cavity 112 when the suture material 20 and suture filament 30 have the same and/or close diameter measurements, as shown in FIG. 11. The advantages of having geometrically shaped X configuration include that as the surgical knot is tighten and partially being pulled towards inside of the cavity 112, it helps to maintain the integrity of the knot as it forms together with the support of the cavity 112; it also assists administering even tension strengths when forming the surgical knot and to avoid the likelihood of a failure knot where it may lead to the danger of untying post operation. The diameters between the suture material 20 and the suture filament 30 is vastly different. For example, if the diameters of the suture material 20 and suture filament 30 are between 0.350 and 0.399 mm and to ensure the surgical knot assembly is efficient for facilitating the formation of a secure surgical knot, then a good corresponding configuration of the assembly would be that the outer tube 10 having a diameter of 2.5-2.6 mm and a length of 23-25 mm, and he inner tube 11 having a diameter of 1.3-1.6 mm and a length of 33-35 mm. Therefore, as the assembly is readily detachable from each other the operator of the assembly may choose and apply the applicable configuration and/or arrangement.

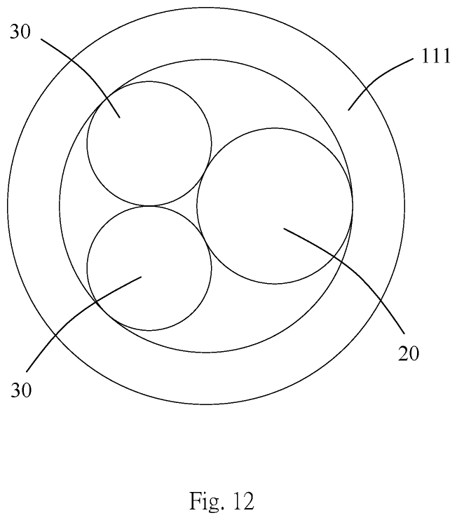

In one embodiment, wherein the suture filament 30 has a much smaller diameter measurements than the suture material 20, as shown in FIG. 12, which makes the geometrically shaped X at the cross-sectional view becoming more like a isosceles triangle.

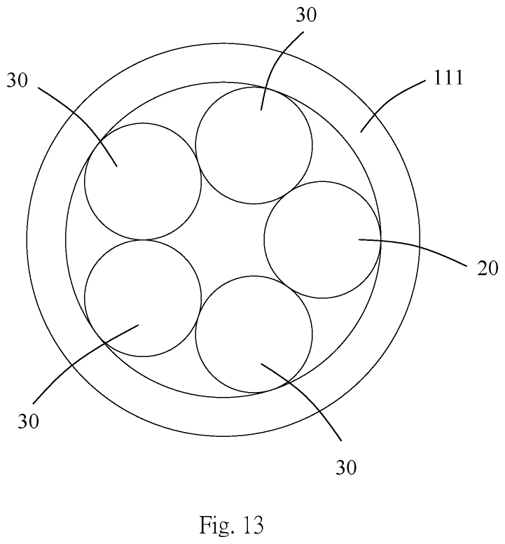

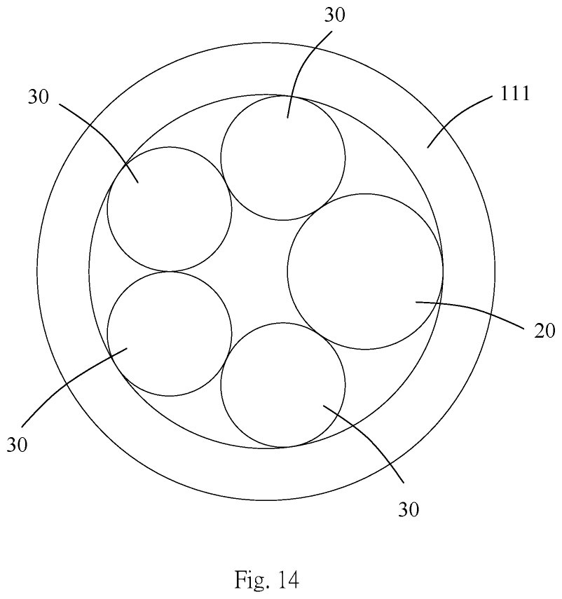

In some embodiments, the cross-sectional view of multiple strands of the suture materials 20 and the suture filaments 30 are pentagonally shaped at the cavity 112 as the suture material 20 and suture filament 30 have the same and/or close diameter measurements, as shown in FIG. 13. In one embodiment, wherein the suture filament 30 has a much smaller diameter measurements than the suture material 20, as shown in FIG. 14.

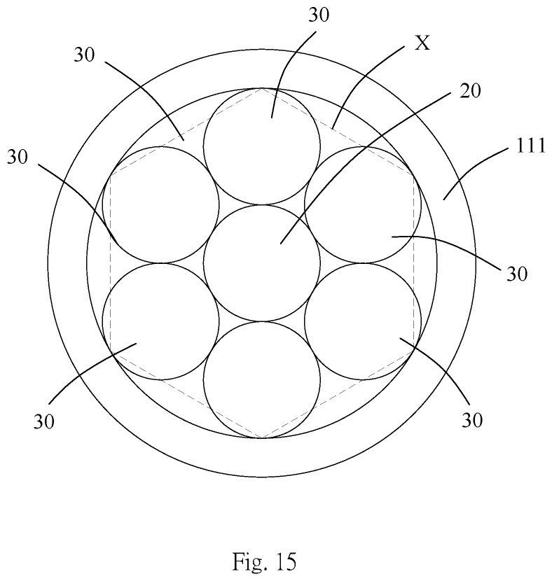

Yet in another embodiment, the cross-sectional view of the multiple strands of the suture materials 20 and the suture filaments 30 are hexagonally shaped at the cavity 112, as shown in FIG. 15.

As the surgical knot A is tightly fastened, diameters of the suture material 20 and filaments 30 could deformed and could be compressed between 3% to 30% for knot security and engagement with the cavity 112. In certain embodiments, the meticulously calculated compression ratio of the suture material 20 and filament 30 and the working combination of the surgical knot assembly is critical for determining the success rate when forming a secure surgical knot each time.

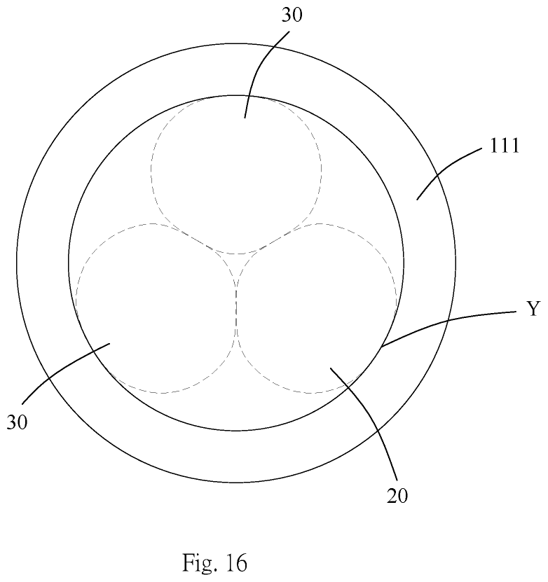

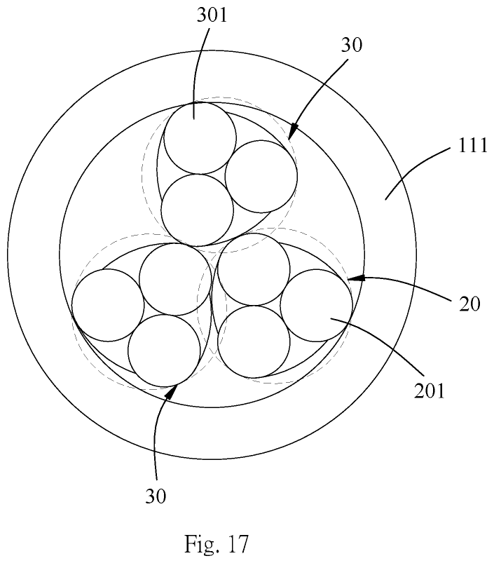

Referring to FIGS. 16 and 17, although there are different embodiments of the surgical knot assembly, but each of the applicable configuration and/or arrangement has its own designs to accommodate the relevant fittings of the suture material 20 and filament 30 for the operation by the operator. For instance, the compression capacity of the suture material 20 and filament 30 at the cavity 112 in the FIG. 16 is 10% and 30%. In some embodiments, monofilament sutures may be easier for handling for some, but it has poorer knot security in comparison to multifilament sutures. The multifilament sutures, e.g., braided sutures 201 and 301, may be preferred by the operator for improved knot security. In certain embodiments, wherein multifilament sutures are used as the suture material 20 and filament 30, and with a two-turn slip pre-tied knot.

Furthermore, the polygonal shape of the cross-sectional view at the cavity 12 could be a closest packing polygon for seeking a certain degree tightness for the suture knot A.

Moreover, the operator would further feel the tactile of tying the knot as the friction and resistance starts to develop and the knot is tightly abutting the walls of the cavity 112 of the inner tube 11. The feedback is further enhanced as the knot is tightly and firmly constructed, wherein at least a side with a curved edge Y abutting the cavity 112 of the inner tube 11.

In light of the aforesaid, the present disclosure and its advantages have been described in detail. Moreover, present disclosure includes several different aspects which addresses or substantially ameliorate one or more the problems disclosed in the specification. It should be understood that various changes, substitutions and alterations can be made herein without departing from the spirit and scope of the disclosure as defined by the appended claims. Other variations will be understood and are included within the scope of the disclosure.

### REFERENCED NUMBER LIST

10 Outer Tube;

11 Inner Tube;

111 Neck;

112 Cavity;

20 A strand of suture material;

201 A braided sutures of the suture material;

21 A pre-tied knot;

22 Loop;

30 A strand of suture filament;

301 A braided sutures of the suture filament;

31 A portion of the suture filament;

311 A tip of the portion of the suture filament;

A A surgical knot;

B Wound site;

X A cross sectional view of a configured surgical knot exhibiting the geometrical arrangements of the pre-tied knot, the loop and the suture filament at the cavity;

Y A cross-sectional view of a configured surgical knot having a curved edge at the cavity.

What is claimed is:

1. A surgical knot assembly for tying a surgical knot, comprising:
an outer tube;
an inner tube slidably interconnected within the outer tube and having a distal end protruding from the outer tube as a neck; and
a strand of suture material having a first portion of a length configured releasably engaged to the neck being a pre-tied knot, and having a second portion of a length extending from a cavity at the distal end of the inner tube, thereby forming a loop between the distal end of the inner tube and the neck to receive at least a strand of suture filament therein,
wherein size of the loop is variable by manoeuvring the suture material and closing the loop as the neck retracts back into the outer tube, allowing the loop, the pre-tied knot and the suture filament to be fastened together forming a surgical knot held at the cavity.

2. The surgical knot assembly according to claim 1, wherein the cross-sectional view of the surgical knot is pentagonally shaped at the cavity.

3. The knotting assembly as claimed in claim 1, wherein the polygonal shape of the cross section of the folded segment is a hexagon.

4. The surgical knot assembly according to claim 1, wherein the pre-tied knot is whirled around the inner tube at least twice and the suture filament intertwined with the loop at least twice when forming the surgical knot.

5. The surgical knot assembly according to claim 4, wherein the suture material and the suture filament are compressed between 3% to 30% when forming the surgical knot.

6. The surgical knot assembly according to claim 4, wherein a partial of the suture material is mounted on the outer tube.

7. The surgical knot assembly according to claim 5, wherein the polygonal shape of the cross-sectional view at the cavity is a closest packing polygon.

8. The surgical knot assembly according to claim 5, wherein the polygonal shape of the cross-sectional view comprises at least a side with a curved edge abutting the cavity of the inner tube

9. The surgical knot assembly according to claim 1, wherein the pre-tied knot is whirled around the inner tube at least twice and the suture filament intertwined with the loop at least twice when forming the surgical knot.
